(19) United States
(12) Patent Application Publication
(10) Pub. No.: US 2023/0112476 A1
(43) Pub. Date: Apr. 13, 2023

(54) Slit Lamp Laminar Airflow Device and Uses Thereof

(71) Applicants: Hellene Coroneo, Randwick, New South Wales (AU); Tomas Hansson, Vasteras (SE)

(72) Inventors: Hellene Coroneo, Randwick, New South Wales (AU); Tomas Hansson, Vasteras (SE)

(21) Appl. No.: 17/913,722

(22) PCT Filed: Mar. 31, 2021
(86) PCT No.: PCT/EP2021/058460

§ 371 Date: Sep. 22, 2022

Related U.S. Application Data
(60) Provisional application No. 63/003,748, filed on Apr. 1, 2020.

(51) Int. Cl. A61G 13/10; F24F 3/163

(52) U.S. Cl. CPC A61G 13/108; F24F 3/163; A61B 90/05

(57) ABSTRACT

The present invention relates to the field of hygienic ophthalmologic examination. In particular, provided is a slit lamp biomicroscope comprising a laminar airflow device, and methods of using the same.

### CROSS REFERENCE TO RELATED APPLICATION

This application claims the benefit of priority from U.S. Provisional Application No. 63/003,748, filed Apr. 1, 2020. The foregoing related application, in its entirety, is incorporated herein by reference.

### FIELD OF THE INVENTION

The present invention relates to the field of hygienic ophthalmologic examination. In particular, provided is a slit lamp biomicroscope comprising a laminar airflow device, and methods of using the same.

### SUMMARY

Provided herein is a slit lamp biomicroscope, comprising: i) a microscope; ii) a light tower; and iii) a laminar airflow device.

Provided herein is a slit lamp biomicroscope, comprising: i) a microscope; ii) a light tower; and iii) a laminar airflow device, and further comprises a mirror.

Provided herein is a slit lamp biomicroscope, comprising: i) a microscope; ii) a light tower; and iii) a laminar airflow device, and further comprises a headrest.

Provided herein is a slit lamp biomicroscope, comprising: i) a microscope; ii) a light tower; and iii) a laminar airflow device, and further comprises one or more deflectors.

Provided herein is a slit lamp biomicroscope, comprising: i) a microscope; ii) a light tower; and iii) a laminar airflow device, and further comprises one or more air aspirators.

Provided herein is a slit lamp biomicroscope, comprising: i) a microscope; ii) a light tower; and iii) a laminar airflow device, and further comprises an laminar airflow generator.

Provided herein is a slit lamp biomicroscope, comprising a laminar airflow device, wherein the laminar airflow device emits laminar air in a downward, vertical trajectory.

Provided herein is a slit lamp biomicroscope, comprising a laminar airflow device, wherein the laminar airflow device emits laminar air in a downward, non-vertical trajectory.

Provided herein is also a method of using a slit lamp biomicroscope, comprising conducting an ophthalmologic examination using a slit lamp biomicroscope as disclosed herein.

Provided herein is also a method of using a slit lamp biomicroscope, comprising conducting an ophthalmologic examination using a slit lamp biomicroscope as disclosed herein, wherein the downward laminar airflow emitted from the laminar airflow device mitigates cross contamination of exhaled air from an examinee and an examiner traversing the slit lamp biomicroscope.

Provided herein is also a method of using a slit lamp biomicroscope, comprising conducting an ophthalmologic examination using a slit lamp biomicroscope as disclosed herein, wherein the method mitigates the risk of spreading a viral infection between a subject being examined and an examiner utilizing the slit lamp biomicroscope to conduct the examination.

### BRIEF DESCRIPTION OF THE DRAWINGS

FIG. 1. Schematic illustrating a slit lamp biomicroscope positioned between an examiner and an examinee during an examination.

FIG. 2. Photograph illustrating a flexible breath shield that is attached to a slit lamp biomicroscope positioned between where an examiner and an examinee would be located.

FIGS. 3A-3B. FIG. 3A: Graphic illustrating the relatively large surface area of the eye(s) was compared to that of the mouth and nares; and FIG. 3B: illustration of the lacrimatory-nasal mechanism for the mechanical disposition of organisms entering the upper respiratory tract.

FIGS. 4A-4B. FIG. 4A: Smoke visualisation of exhalation flow from mouth of manikin on the right, wherein the two manikins are separated by a distance of 1.2 m; FIG. 4B: Smoke visualisation of exhalation flow from nose of manikin on the right, wherein the two manikins are separated by a distance of 0.4 m.

FIGS. 5A-5B. FIG. 5A: Schematic illustrating a slit lamp biomicroscope comprising a laminar airflow device emitting a laminar airflow between an examiner and an examinee during an examination, wherein airflow emitted from the laminar airflow device is in a downward trajectory between the examiner and the examinee. FIG. 5B: Schematic illustrating a slit lamp biomicroscope comprising a laminar airflow device, wherein the laminar airflow device is positioned proximate to the examinee headrest, such that the airflow emitted between an examiner and an examinee during an examination is proximate and between the examiner and the examinee.

FIG. 6. Schematic illustrating a slit lamp biomicroscope comprising a laminar airflow device emitting a laminar airflow between an examiner and an examinee during an examination, wherein the slit lamp biomicroscope is further fitted with one or more air deflectors between the examiner and the examinee.

FIG. 7. Schematic illustrating a slit lamp biomicroscope comprising a laminar airflow device emitting a laminar airflow between an examiner and an examinee during an examination, wherein the slit lamp biomicroscope is further fitted with one or more air aspirators between the examiner and the examinee.

FIG. 8. Schematic illustrating a slit lamp biomicroscope comprising a laminar airflow device emitting a laminar airflow between an examiner and an examinee during an examination, wherein the laminar airflow device is emitting cold air between the examiner and the examinee.

FIG. 9. Schematic illustrating a slit lamp biomicroscope comprising a laminar airflow device emitting a laminar airflow between an examiner and an examinee during an examination, wherein the laminar airflow device is emitting the laminar airflow air in a single downward, non-vertical, trajectory between the examiner and the examinee.

FIG. 10. Schematic illustrating a slit lamp biomicroscope comprising a laminar airflow device emitting a laminar airflow between an examiner and an examinee during an examination, wherein the laminar airflow device is emitting the laminar airflow air in at least two different downward, non-vertical, trajectories between the examiner and the examinee.

FIG. 11. Schematic illustrating a slit lamp biomicroscope comprising a laminar airflow device emitting a laminar airflow between an examiner and an examinee during an examination, wherein the slit lamp biomicroscope is further fitted with a nozzle associated with a fan and/or filter system that removes the emitted laminar air flowing between the examiner and the examinee from the laminar airflow device.

FIG. 12. Schematic illustrating an air cleaning device, comprising an air cleaning screen (a), a fan (b), and an air cleaning filter (c).

FIG. 13. Schematic illustrating a slit lamp biomicroscope enclosed with a drape or hood enclosure, wherein the enclosed slit lamp biomicroscope comprises a laminar airflow device emitting a laminar airflow within the enclosure such that the emitted laminar air is then removed via a nozzle associated with a fan or filter (or filter device having a fan and filter), or alternatively comprises an air cleaning device emitting clean airflow within the enclosure such that the emitted clean air is then removed via a nozzle associated with a fan or filter (or filter device having a fan and filter); and wherein the examiner is able to access the slit lamp biomicroscope via an opening (Examiner's viewpoint) in the drape or hood enclosure and the examinee is able to access the headrest via an opening in the drape or hood enclosure for the examinee's entire face (or alternatively one opening for the eyes and a separate opening for the examinee's nose and mouth).

### DETAILED DESCRIPTION

As used herein, the articles “a,” “an,” and “the” refer to one or to more than one of the grammatical object of the article. By way of example, a sample refers to one sample or two or more samples.

As used herein, the term “subject” refers to a mammal. A subject can be a human or a non-human mammal such as a dog, cat, bovid, equine, mouse, rat, rabbit, or transgenic species thereof. The subject may have been exposed to a microbe, a bacteria, a virus, such as a coronavirus, for example, COVID-19 virus, or such as an adenovirus or a rhinovirus, for example, influenza virus. In some embodiments, the subject may have a bacterial infection or a virus infection, such as a coronavirus infection, for example, COVID-19 infection, or such as a rhinovirus infection, for example, influenza infection. The subject can be a patient who has a COVID-19 infection (e.g., has been diagnosed with a COVID-19 infection), or the subject can be a patient who has been exposed to a COVID-19 virus, but does not show one or more symptoms of having a COVID-19 infection. An examinee can be a mammal, for example a human. An examiner can be a human, such as a person trained in using a slit lamp biomicroscope, for example, doctor, such as an ophthalmologist.

The nature of ophthalmic practice has traditionally involved very close physical proximity of the ophthalmologist to the patient. This is necessitated by optical imperatives to optimize image quality by close alignment of the imaging device to the object of regard—very close in the case of direct ophthalmoscopy and at a slightly greater distance for slit lamp biomicroscopy. Evolution of imaging techniques has meant that direct ophthalmoscopy is in general used less, since high quality retinal imaging can be carried out by slit lamp-assisted indirect ophthalmoscopy and other retinal imaging methodologies which place the examiner even further away from the eye. This has meant that a cornerstone of ophthalmic practice remains use of the slit lamp.

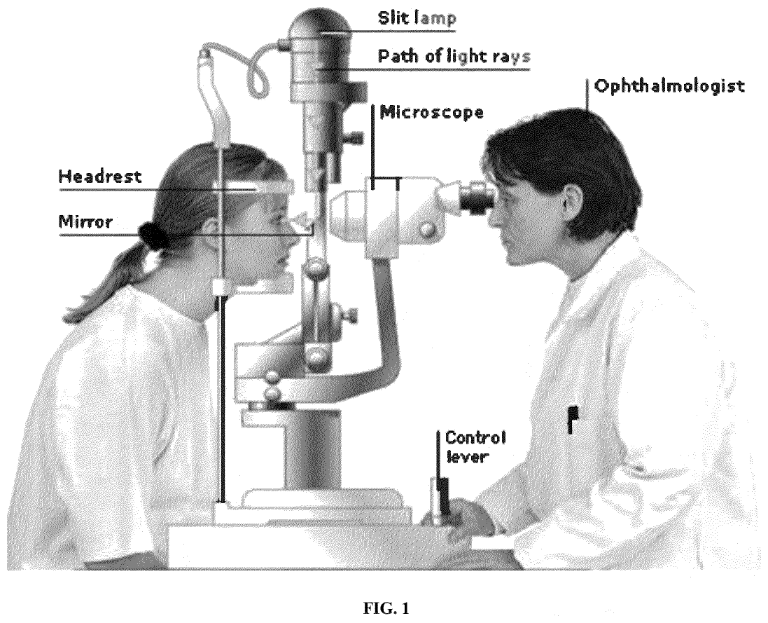

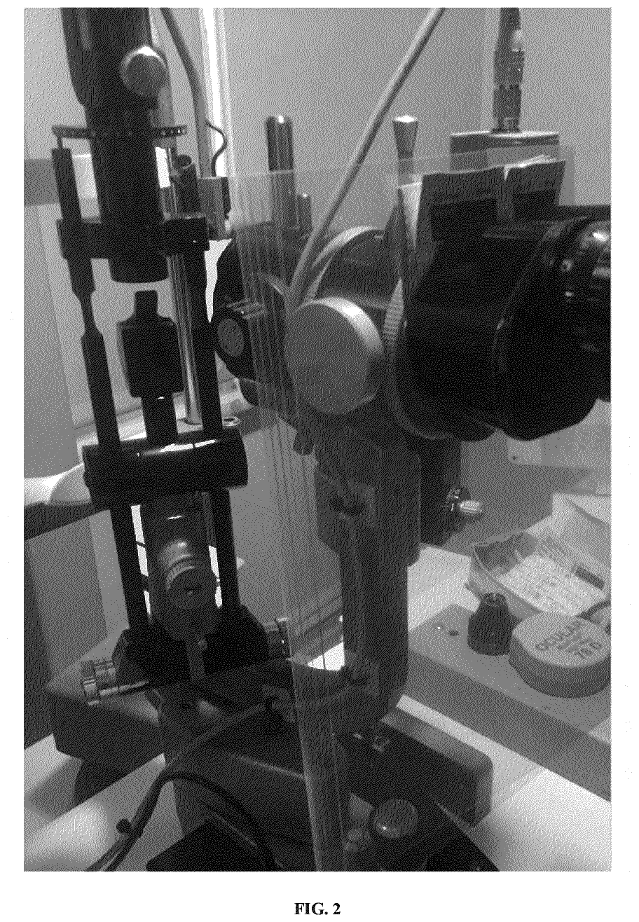

Apart from imaging, the slit lamp biomicroscope (also referred to herein as the “slit lamp,” “biomicroscope”, or “microscope”) is also used to carry out surgical procedures, so that the back focal distance (the distance of the subject from the front lens surface of the microscope), must have a certain minimum length to give the surgeon sufficient space for manipulation. If it is too long, ocular procedures are difficult, because of the resulting extended and uncomfortable position of the surgeon's arms. Illustrated in FIG. 1, is a schematic of a slit lamp biomicroscope that is positioned between an examiner (e.g., an ophthalmologist) and an examinee (e.g., a patient) during an examination, which shows the positioning of the microscope relative to the light-tower that houses the slit lamp and directs the path of light rays towards a mirror positioned proximate to the examinee's eyes, whose head is positioned securely in a headrest. In some embodiments, the recommended back focal distance of a slit lamp is in the range of between about 9-12 cm (1) (a rule of thumb is a back focal distance of about 11 cm). The biomicroscope body length is about 17 cm, any may be longer in modern slit lamps when attachments, such as a beam splitter, are added. Thus, in some embodiments, the distance from the patient's eye to the surgeon's eye is at a convenient working distance in the range of between about 28-30 cm. However, the possibility of cross infection between the examiner and the examinee has long been recognized as a risk resulting from this proximity. A protective, transparent, typically PERSPEX, “breath shield” (sometimes described as a “hygiene accessory”) was added to slit lamps to create a rigid, physical barrier between the examiner (e.g., a doctor, an ophthalmologist, an optometrist, a surgeon) and the examinee (e.g., a subject, such as a patient) so as to protect against this risk. It is uncertain when this breath shield was introduced and the events that precipitated its introduction. Similarly, a rigid, physical breath shield for the direct ophthalmoscope was described in 1955 (2) but did not enter into everyday practice. The rigid, physical breath shield is typically about 14×11 cm in size and is attached to the microscope such that it is positioned between the examiner and the examinee. It is often absent from the microscope (generally provided only as an “accessory”). More recent make-shift breath shields made from flexible plastic sheets (as shown attached to the slit lamp biomicroscope in FIG. 2; sometimes referred to herein as a “flexible, physical breath shield” or “flexible breath shield”), have the advantage that their flexibility allows for better access to the patient and to slit lamp accessories, such as the applanation tonometer. However, while the flexible, physical barrier created by the flexible breath shield reduces direct flow of contaminated breath being exchanged between the examiner and the examinee, it is not typically large enough to stop all flow, as this would prevent manual access to the examinee. Furthermore, regular, hygienic cleaning of the screen is required and the reflected breath on each side, can result in particles, such as microbes, bacteria, or viruses, landing on and adhering to the microscope instrument.

In light of the recent coronavirus (COVID-19) pandemic, focus is being brought to bear on ophthalmic aspects of this disease. Ophthalmology is perhaps uniquely exposed to the possible impacts of COVID-19 (3): “Our mostly geriatric patients are the most susceptible to dying from the virus. And the typical ophthalmologist works nose-to-nose with 150 patients a week. Before long, some of those noses could be loaded.” The mortality rate in this pandemic may be as high as 20% (4) and it transpires that ophthalmologists along with ENT surgeons are at particular risk (5). It has been reported that a significant number of doctors who died in China were ophthalmologists and ENT surgeons, and that this was attributed to high viral shedding from the nasal cavity of the respective patients (5,6).

COVID-19 aerosol and fomite transmission is may be high, since this coronavirus is reportedly able to remain viable and infectious in aerosols for hours and on surfaces for up to days (7). Other viruses, such as rhinoviruses, for example, the infectious influenza virus (both as fine- and coarse-aerosol viral RNA), have been found in the exhaled breath of symptomatic seasonal influenza patients (8). According to this study, sneezing was rare, and sneezing and coughing were not necessary for infectious aerosol generation (8).

Slit lamps and their accessory lenses have been identified as a potential source of bacterial infection (9) and the microscope itself (and its components) is known to be a means of spreading infectious viral conjunctivitis (9). Other infectious transmissions, such as adenoviral infection transmission in eye clinics/hospitals has long been known (10)—epidemic adenoviral keratoconjunctivitis (shipyard eye), also known as “eye hospital eye” (11). Although not well documented, ophthalmologists are not infrequently infected (12). While viral infections generally can cause considerable incapacity and has resulted in some risk-minimization measures, the recent reports relating to mortality rates associated with coronavirus infections, including COVID-19 infections, exposes the need to improve these risk-minimization measures. For example, the earlier severe acute respiratory syndrome (SARS) epidemic resulted in recommendations for how to manage eye facilities and include advice in relation to slit lamp cleaning and eye protection for staff (13) and this has been more recently reiterated (14). Yet, given the proximity between the examiner and the examinee, the use of slit lamp biomicroscopy may be an optimal method of transferring material in breaths between two individuals, short of actual facial contact. Such proximity during the use of a slit lamp biomicroscopy may also increase the chance of contracting a higher initial viral load from an infected individual (either the examiner or the examinee), which may lead to an increased severity of an eventual viral infection (27). Cleaning all of the parts of the slit lamp is often recommended, but compliance can be very difficult, and the repeated use of cleaning agents may damage optical components. Moroever, the examiner and the examinee (e.g., the patient and doctor) are breathing in the same plane and are in close proximity (at least 6 times closer than the recently recommended “social distancing” distance to be maintained between individuals of at least 6 ft. to minimize the spread of a viral infections, such as spread of the COVID-19 infection), thereby greatly increasing the risk of infections, such as viral infections, between the examiner and the examinee, irrespective of using a clean slit lamp.

We have recently proposed that the major portal of coronavirus entry into the body is via the eyes and periocular tissue, with the virus either binding directly to the ocular surface or being carried via the tear film into the nasopharynx and thence into the lungs and gastrointestinal tract (15).

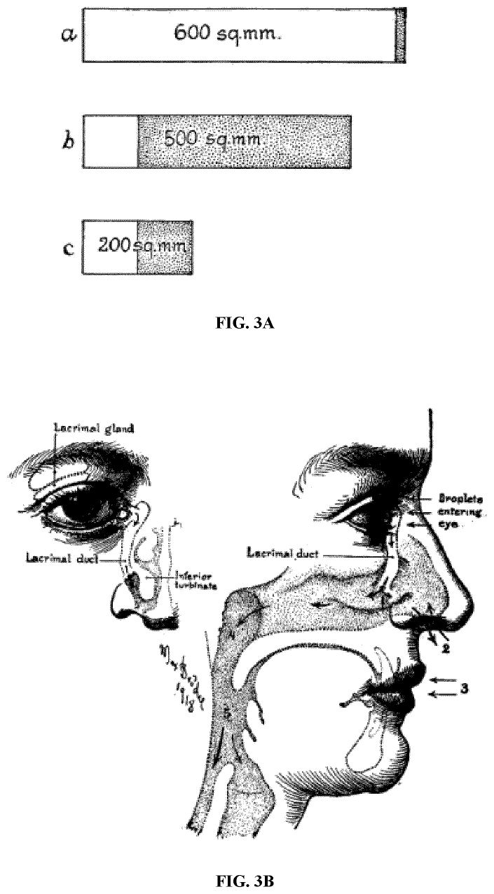

The relative importance of eye exposure to direct droplet spray, is brought into focus when considering the relatively large surface area of the eye(s) when compared to that of the mouth and the nares, as illustrated FIG. 3A (wherein (a) represents average total eye surface exposed; shaded area represents proportion of time not exposed, due to winking; (b) represents average total mouth area exposed in talking; shaded area represents proportion of time not exposed, due to closure; and (c) represents average total area of cross-section of nares exposed; shaded area represents proportion of time not exposed, owing to protected position and expiration) (16). In the 1919 study (16), Bacillus prodigiosus (Serratia marcescens) was instilled into the lacrimal sac of 5 volunteers and subsequently was recoverable from the nose, throat and stool after 5 mins, 15 mins and 24 hours respectively. It was concluded that via this ocular lacrimatory-nasal mechanism (shown in FIG. 3B, excerpted from (16)), that the upper respiratory tract of a person wearing a properly constructed mask may be infected by exposing the eye briefly to a direct droplet spray (16).

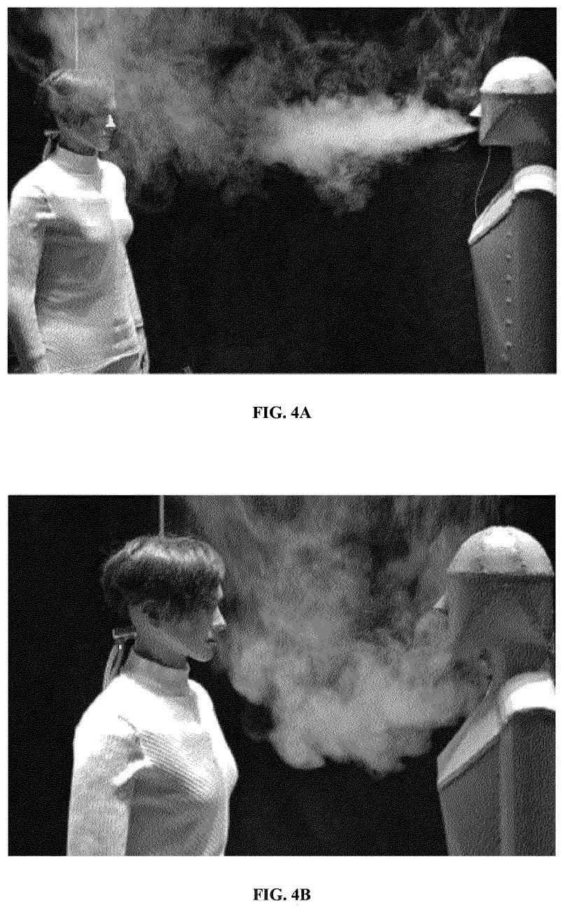

The concept of droplet spray infection originates from the findings of Flugge in 1897, that expiratory droplets contained bacteria and could not travel more than 1-2 m (17). Human expiratory droplets are produced by the atomization of human secretions along the airway (18). Large droplets may travel up to 1.5 m, so transmission and effectiveness of masks against large droplet transmission, can occur within this range (19,20)—also referred to as the “proximity effect.” The acts of breathing, coughing, and sneezing releases thousands of droplets per respiration, with a wide spectrum of dropet sizes ranging from submicrons to millimetres in diameter size (19). Post exhalation, droplets may evaporate and become droplet nuclei (21), and the size may depends on droplet composition and surrounding humidity (19,22). Of relevance to slit lamp biomicroscopy, are studies in which exposure between two face-to-face breathing thermal manikins in a ventilated room was measured (23). In these experiments, as illustrated in FIGS. 4A-4B, the two manikins faced each other and one manikin exhales either through the mouth (FIG. 4A) or the nose (FIG. 4B) (23). Tracer gas was added to the exhalation of the manikin on the right and the distance between the manikins was 1.2 m (FIG. 4A) and 0.4 m (FIG. 4B), measured from mouth to mouth, respectively. In FIG. 4A, the smoke visualisation of exhalation flow from mouth of manikin on the right, and the breathing zone of manikin A (on the left, wearing white shirt) is penetrated by the mouth exhalation flow despite a separation distance of 1.2 m. In FIG. 4B, the smoke visualisation of exhalation flow from nose of the manikin on the right, and the breathing zone of manikin A (on the left, wearing white shirt) is penetrated by the nose exhalation flow despite a separation distance of 0.4 m. Extrapolating these results to the approximately 30 cm slit-lamp working distance situation, under either instance, whether via mouth or nose breathing, both the examiner and the examinee are within the “line of fire” of their counterparts breathing, and thus each is at risk for contracting an infection, such as a viral infection, if one of the individuals has a viral infection, or has been recently exposed to a virus. This study concludes that air exhaled from human respiration (both nose and mouth) contains contaminants, and is able to penetrate the breathing zone of other persons located nearby (23), which further exemplifies the relatively high degree of risk that both the examiner and examinee are exposed to when considered in the context of the slit-lamp working distance situation. Of interest is that exhalation flow may stratify in a horizontal layer at breathing zone height under certain conditions (23). Thus, for example, during a slit lamp examination—separated by slit-lamp working distance, the layer of air between a patient and an ophthalmologist may remain stable, and may expose both individuals for a majority of the time, or possibly the entire time, of the examination.

Efforts have been made using ultraclean laminar airflow to reduce/prevent surgical site infections (SSIs). A systematic review demonstrated no benefit for laminar airflow compared with conventional turbulent ventilation of the operating room in reducing the risk of SSIs in orthopaedic and abdominal surgery (25). However, a subsequent study reported that if average airborne concentrations of microbe-carrying particles (“MCPs”) are kept less than 10/m³, and preferably less than 1/m³, then deep joint infection after total joint arthroplasty (“TJA”) will be lower than in conventionally ventilated operating theatres (25). A device known as the SurgiCube®, developed for ophthalmic surgery, complies with this higher standard and has been reported to be an efficient airflow device capable of minimizing the spread of airborne contamination, including the latest coronavirus SARS-CoV-2 (26).

A vertical ultraclean laminar airflow attachment (a laminar airflow device), has been produced specifically for the slit-lamp. The laminar airflow device is attached to existing slit lamp structures, either the head rest or the light tower. It encompasses the extent of the instrument and both the patients' and examiners' faces. The downdraft of air, which may be silent, cold, or filtered, or combinations thereof, may mitigate, such as block, for example completely block, exhaled air from both the patient and the examiner traversing the instrument, reducing the risk of cross contamination in this setting, and approaching or even achieving the absence of risk of cross contamination. Use of the slit lamp biomicroscope comprising a laminar airflow device, as disclosed herein, may further minimize the risk of breath-laden contaminants from adhering to the instrument.

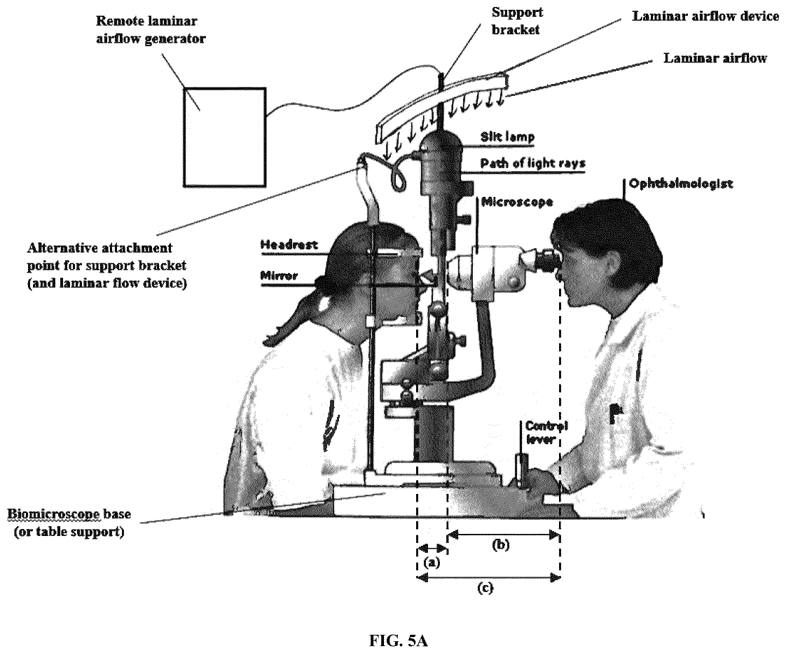

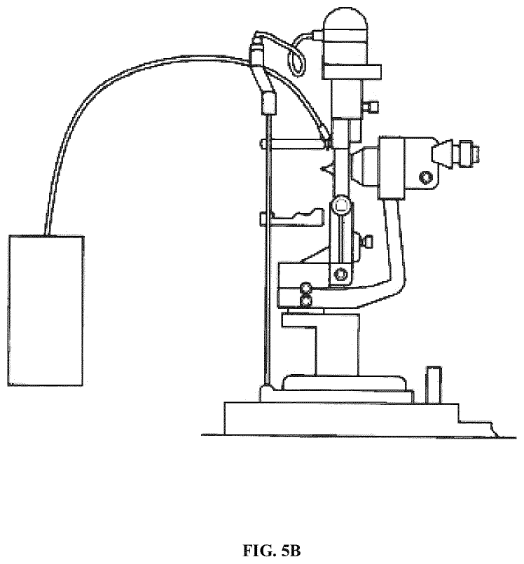

Illustrated in FIG. 5A is a schematic showing a slit lamp biomicroscope positioned between an examiner and an examinee during an examination, wherein a laminar airflow device is attached to the slit lamp biomicroscope via a support bracket, and wherein airflow is provided to the slit lamp biomicroscope via a remote laminar airflow generator, and airflow emitted from the laminar airflow device is in a downward trajectory between the examiner and the examinee, such as in a vertical, downward trajectory. As shown in FIG. 5A, distance (a) between an examinee (e.g., a patient) and the lens of the microscope when the examinee's head is secured in the headrest is about 9-12 cm (1) (a rule of thumb is a back focal distance of about 11 cm), and the distance (b) is the biomicroscope body length, which is about 17 cm, but may be longer in when attachments, such as a beam splitter, are added, and the disctance (c) between an examiner (e.g., an ophthalmologist) and an examinee (e.g., a patient) during an examination, as measured from the examinee's eye to the examiner's eye is in the range of between about 28-30 cm. Also illustrated in FIG. 5A, is an alternative attachment point at the head rest for mounting the laminar airflow device to slit lamp biomicroscope. Illustrated in FIG. 5B is a schematic showing one possible variation of the slit lamp biomicroscope positioned between where an examiner and an examinee would be located during an eye examination (as shown in FIG. 5A), but here the laminar airflow device is positioned proximate to the examinee headrest, such that the airflow emitted between an examiner and an examinee during an examination is proximate and between the examiner and the examinee.

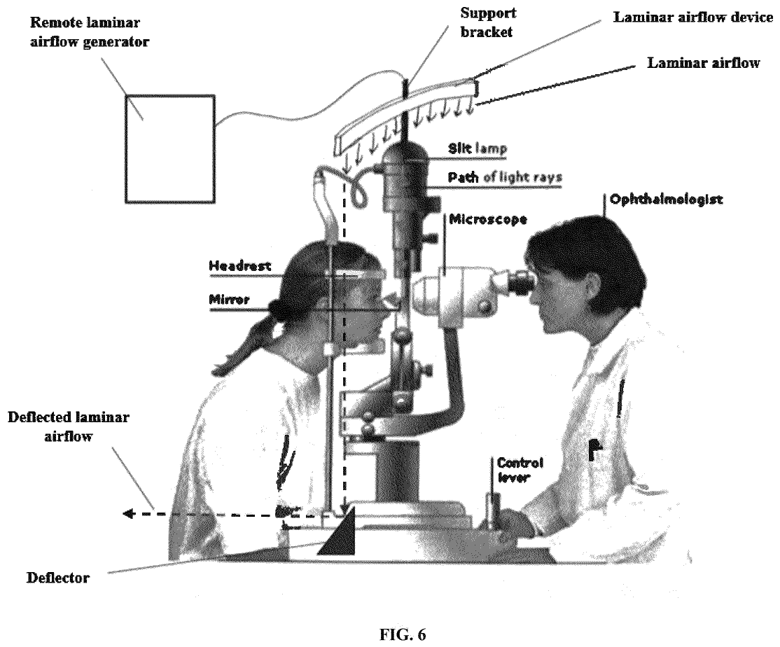

Illustrated in FIG. 6 is a schematic showing a variation of the slit lamp biomicroscope shown in FIG. 5A, wherein the slit lamp biomicroscope is further fitted with one or more air deflectors between the examiner and the examinee, which deflects the emitted air from the laminar airflow device at a non-vertical angle so that the emitted air does not bounce off the surface of the microscope base or table support back into the breathing zone of either the examinee or the examiner.

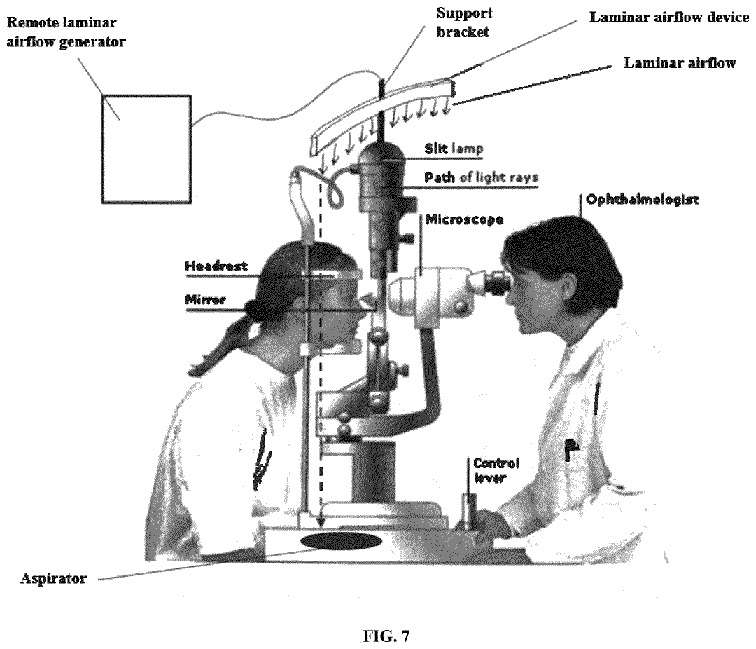

Illustrated in FIG. 7 is a schematic showing a variation of the slit lamp biomicroscope shown in FIG. 5A, wherein the slit lamp biomicroscope is further fitted with one or more air aspirators between the examiner and the examinee, which removes the emitted air from the laminar airflow device so the emitted air does not remain or enter the breathing zone of either the examinee or the examiner.

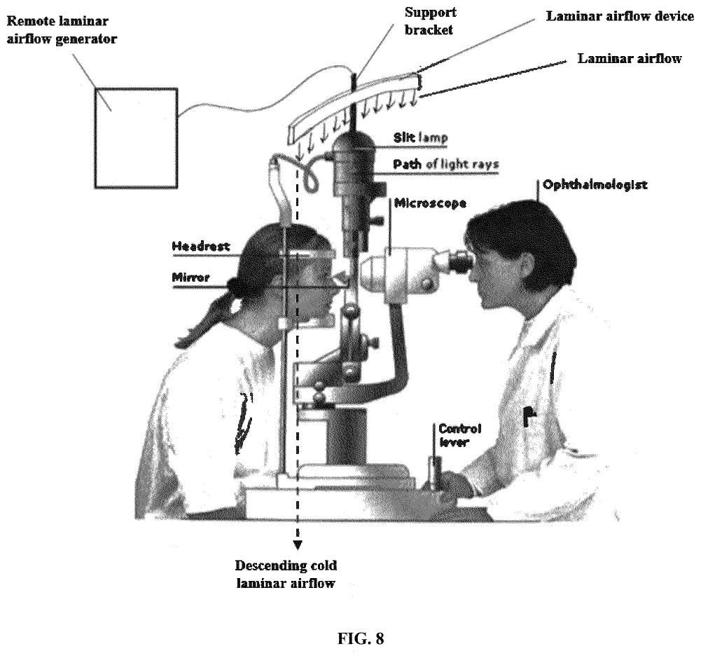

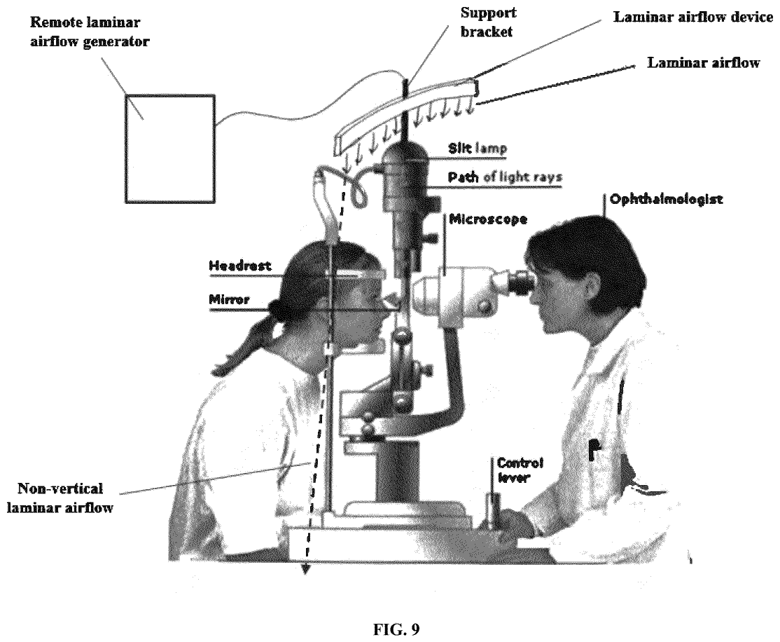

Illustrated in FIG. 8 is a schematic showing a variation of the slit lamp biomicroscope shown in FIG. 5A, wherein the laminar airflow device is shown emitting cold air between the examiner and the examinee, which has a tendency to remain lower than warm air, and thus decreases the risk of the emitted air from the laminar airflow device remaining or entering the breathing zone of either the examinee or the examiner.

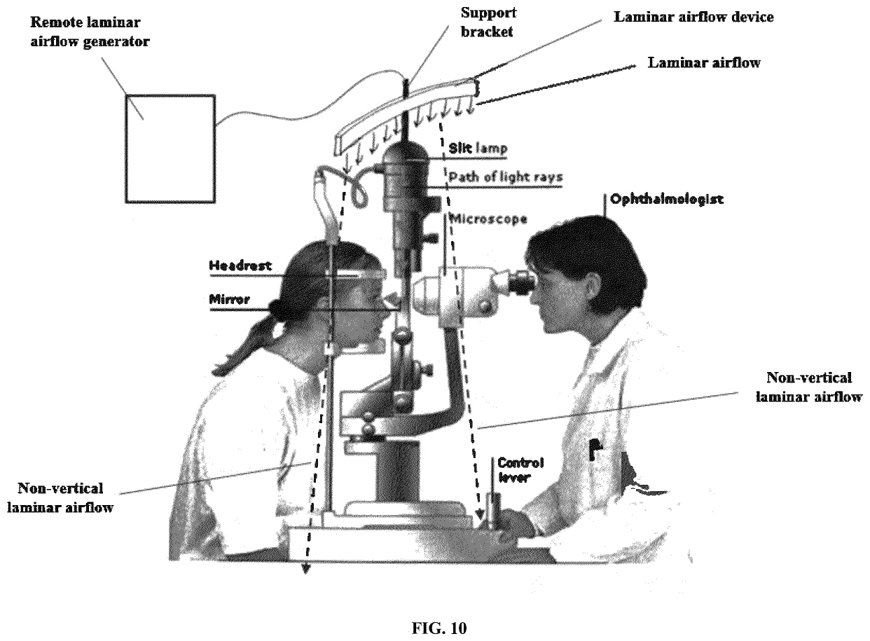

Illustrated in FIG. 9 is a schematic showing a variation of the slit lamp biomicroscope shown in FIG. 5A, wherein the airflow emitted from the laminar airflow device is in a downward, non-vertical, trajectory between the examiner and the examinee, which decreases the risk of cross contamination of the breathing zones of either the examinee and the examiner. In some embodiments, the downward, non-vertical, trajectory of the airflow emitted from the laminar airflow device is in a single direction, such at an angle towards the examinee, or at an angle towards the examiner. As illustrated in FIG. 10, the airflow emitted from the laminar airflow device is emitted in at least two different downward, non-vertical, trajectories between the examiner and the examinee, such as a first downward, non-vertical, trajectory at an angle towards the examinee, and a second downward, non-vertical, trajectory at an angle towards the examiner, thereby mitigating or eliminating the risk of cross contamination of the breathing zones of either the examinee and the examiner. In some embodiments, the slit lamp biomicroscope disclosed herein is a combination of one or more of the embodiments illustrated in FIGS. 5-10.

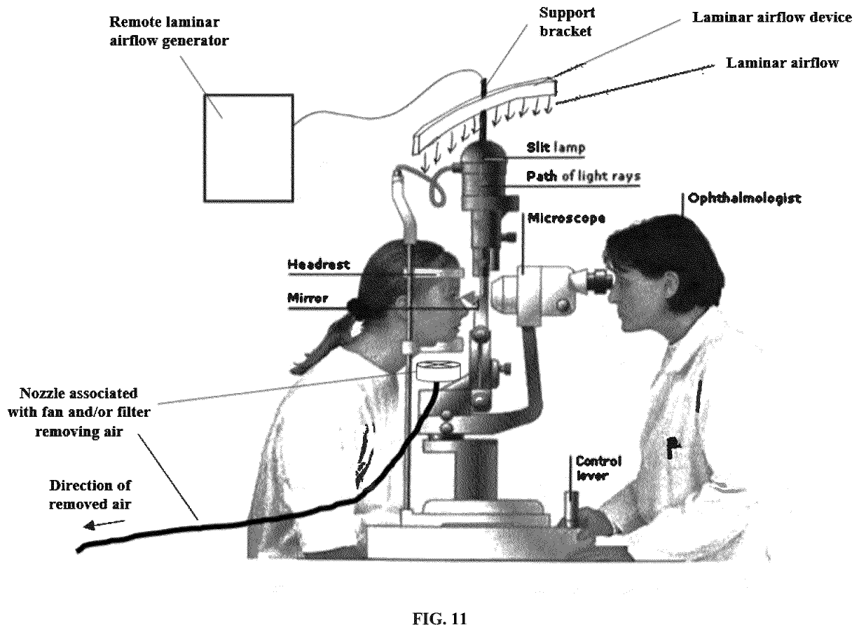

Illustrated in FIG. 11 is a schematic showing a slit lamp biomicroscope comprising a laminar airflow device emitting a laminar airflow between an examiner and an examinee during an examination, wherein the slit lamp biomicroscope is further fitted with a nozzle associated with a fan and/or filter system, such as an air cleaning device (comprising an air cleaning screen, a fan, and/or an air cleaning filter) or an air cleaning filter device (comprising a fan and air cleaning filter) that removes the emitted laminar air flowing between the examiner and the examinee from the laminar airflow device. In some embodiments, the nozzle associated with a fan and/or filter system is connected to a pump or suction device to remove the air emitted from the laminar airflow device and the air in the are between the examiner and the examinee.

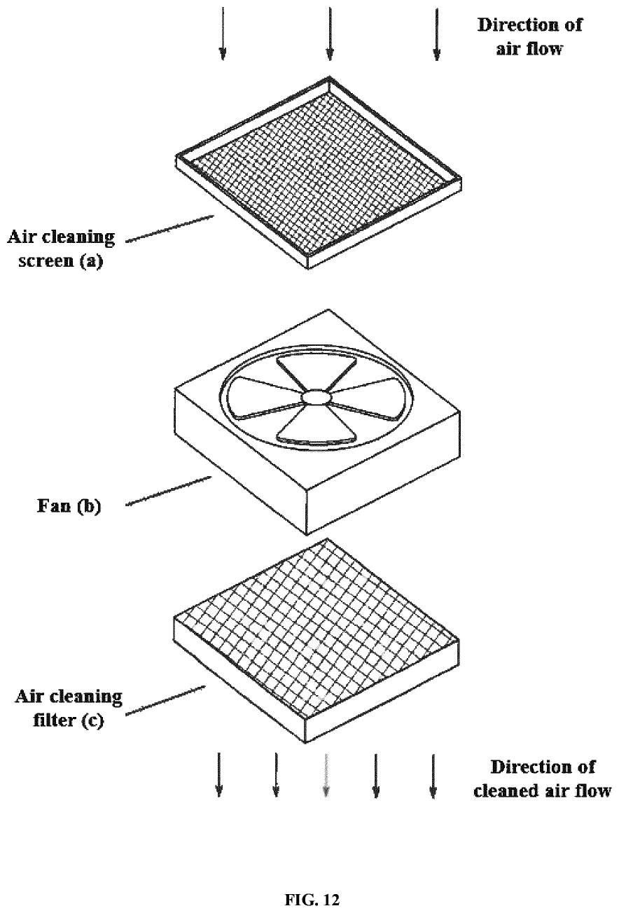

Illustrated in FIG. 12 is a schematic showing an air cleaning device, comprising an air cleaning screen (a), a fan (b), and an air cleaning filter (c). In some embodiments, the air cleaning device is the air cleaning screen. In some embodiments, the air cleaning device is the air cleaning screen and the an air cleaning filter. In some embodiments, the air cleaning device is the air cleaning screen, the fan, and the an air cleaning filter. In some embodiments, the filter is a HEPA filter. In some embodiments, the filter excludes (filters) particles having a diameter of about 0.01-0.20 um or greater, such as particles having a diameter of about 0.05-0.15 um or greater. In some embodiments, the filter excludes (filters) airborne particles comprising bacteria and/or viruses, such as coronaviruses.

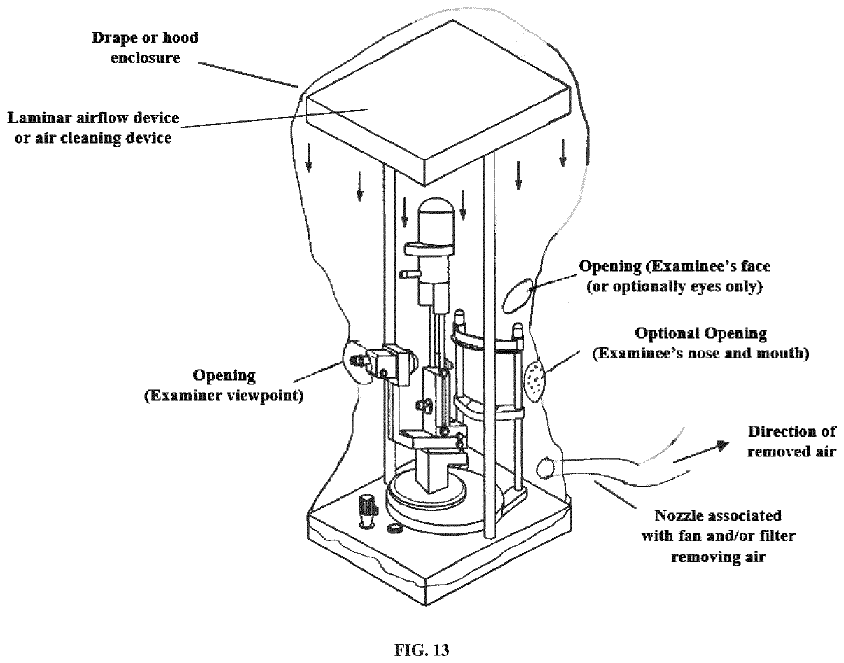

Illustrated in FIG. 13 is a schematic showing a slit lamp biomicroscope enclosed with a drape or hood enclosure, wherein the enclosed slit lamp biomicroscope comprises a laminar airflow device emitting a laminar airflow within the enclosure such that the emitted laminar air is then removed via a nozzle associated with a fan or filter (or filter device having a fan and filter), or alternatively comprises an air cleaning device emitting clean airflow within the enclosure such that the emitted clean air is then removed via a nozzle associated with a fan or filter (or filter device having a fan and filter); and wherein the examiner is able to access the slit lamp biomicroscope via an opening (Examiner's viewpoint) in the drape or hood enclosure and the examinee is able to access the headrest via an opening in the drape or hood enclosure for the examinee's entire face (or alternatively one opening for the eyes and a separate opening for the examinee's nose and mouth). In some embodiments, the drape or hood enclosure is transparent. In some embodiments, the enclosed slit lamp biomicroscope is fitted with a laminar airflow device and an air cleaning device connected to a pump or suction device to remove the air emitted from the laminar airflow device.

In some embodiments, an eye examination equipment is provided comprising: a head rest against which a patient whose eye will be examined can rest his/her forehead; and a microscope positioned such that an eye of the patient can be examined through the microscope. In some embodiments, the eye examination equipment further comprises: an air cleaning screen which is positioned and configured for transferring a flow of air either by providing an outflow of air from the air cleaning screen such that the outflow of air passes between the head rest and the microscope or by providing a suction of air from an area between the head rest and the microscope into the air cleaning screen, wherein said air cleaning screen is configured to be connected to a filter device for cleaning of the air. In some embodiments, the eye examination equipment further comprises a slit lamp, wherein the eye examination equipment is a slit lamp microscope with a connected air cleaning screen. In some embodiments, the eye examination equipment comprises the filter device, which is mounted together with the air cleaning screen on a stand provided in the eye examination equipment. In some embodiments, the air cleaning screen is configured to be connected to the filter device by a tube. In some embodiments, the filter device comprises a HEPA filter and a fan. In some embodiments, the filter device comprises a UVC device. In some embodiments, the air cleaning screen is mounted to a slit lamp provided in the eye examination equipment, or is mounted to a stand provided in the eye examination equipment. In some embodiments, the air cleaning screen is mounted onto the head rest. In some embodiments, the drape is a disposable drape, for example, a transparent pouch for eye examination equipment. In some embodiments, the drape can be pulled over the equipment to prevent airborne particles such as bacteria and viruses from being moved between doctors and patients and to prevent contact contamination on the equipment. In some embodiments, the drape has holes for the microscope and an opening for the patient's eyes. In some embodiments, the drape can be connected to a pump with Hepa filter for easy suction suppression and to suck in dangerous particles. In some embodiments, the drape can be provided with many small holes at the nose and mouth to suck in particles. In some embodiments, a fixed transparent cover (e.g., a hood enclosure) can protect the equipment, wherein the cover may be provided with a filter air device at its top and a suction device at its bottom, and have openings for microscopes and the patient's eyes and nose and mouth, but at a distance so that the patient never come in contact with his face against the cover.

In some embodiments, the slit lamp biomicroscope as disclosed herein, and methods of using the same, is suitable for reducing exposure to microbes, and protecting against contaimination or infection from microbes, between the examinee and/or the examiner during an examination. In some embodiments, the eye examination equipment as disclosed herein, and methods of using the same, is suitable for reducing exposure to microbes, and protecting against contaimination or infection from microbes, between the examinee and/or the examiner during an examination. In some embodiments, the microbes include bacteria and/or viruses, such as coronaviruses, for example COVID-19, or rhinoviruses, for example, influenza.

It is understood that subheadings throughout this document do not limit the subject matter discussed to only those sections, but apply, and are contemplated to apply, to each embodiment disclosed in the instant application.

It is understood that modifications which do not substantially affect the activity of the various embodiments of this invention are also provided within the definition of the invention provided herein. Accordingly, the following examples are intended to illustrate but not limit the present invention. All of the references cited to herein are incorporated by reference in their entireties.

### EXAMPLES

### Exemplary Embodiments

A1. In an embodiment, a slit lamp biomicroscope, comprising:
i) a microscope;
ii) a light tower; and
iii) a laminar airflow device.

A2. In an embodiment, a slit lamp biomicroscope, comprising:
i) a microscope;
ii) a light tower;
iii) an air cleaning device; and
iv) a drape or hood enclosure surrounding the slit lamp biomicroscope.

In certain embodiments, one or more than one (including for instance all) of the following further embodiments may comprise each of the other embodiments or parts thereof.

A3. The slit lamp biomicroscope of embodiment A1 or embodiment A2, wherein the slit lamp biomicroscope further comprises a mirror.

A4. The slit lamp biomicroscope of any one of embodiments A1-A3, wherein the slit lamp biomicroscope further comprises a headrest.

A5. The slit lamp biomicroscope of embodiment A4, wherein the headrest is suitable for a patient to place their head against during an eye examination or procedure.

A6. The slit lamp biomicroscope of any one of embodiments A1-A5, wherein the slit lamp biomicroscope further comprises a biomicroscope base.

A7. The slit lamp biomicroscope of embodiment A6, wherein the biomicroscope base comprises one or more deflectors.

A8. The slit lamp biomicroscope of embodiment A6 or embodiment A7, wherein the biomicroscope base comprises one or more air aspirators.

A9. The slit lamp biomicroscope of any one of embodiments A1-A8, wherein the slit lamp biomicroscope is mounted to a table support.

A10. The slit lamp biomicroscope of embodiment A9, wherein the table support comprises one or more deflectors.

A11. The slit lamp biomicroscope of embodiment A9 or embodiment A10, wherein the table support comprises one or more air aspirators.

A12. The slit lamp biomicroscope of any one of embodiments A7-A11, wherein the one or more deflectors deflect laminar airflow emitted from the laminar airflow device.

A13. The slit lamp biomicroscope of any one of embodiments A7-A12, wherein the laminar airflow deflected by the one or more deflectors is angled towards an examinee positioned at the headrest.

A14. The slit lamp biomicroscope of any one of embodiments A7-A13, wherein the laminar airflow deflected by the one or more deflectors is angled towards an examiner positioned looking through the microscope to examine an examinee positioned at the headrest.

A15. The slit lamp biomicroscope of any one of embodiments A7-A14, wherein a first portion of the deflected laminar airflow is angled towards an examinee positioned at the headrest, and a second portion of the deflected laminar airflow is angled towards an examiner positioned looking through the microscope to examine an examinee positioned at the headrest.

A16. The slit lamp biomicroscope of any one of embodiments A8-A15, wherein the one or more air aspirators remove the emitted laminar airflow.

A17. The slit lamp biomicroscope of any one of embodiments A8-A16, wherein the one or more air aspirators remove the emitted laminar airflow and transmit it towards a filtering device.

A18. The slit lamp biomicroscope of any one of embodiment A1 or embodiments A3-A17, wherein the slit lamp biomicroscope further comprises an air cleaning device.

A19. The slit lamp biomicroscope of embodiment A18, wherein the air cleaning device is located proximate to the headrest.

A20. The slit lamp biomicroscope of embodiment A18 or embodiment A19, wherein the air cleaning device comprises an air cleaning screen.

A21. The slit lamp biomicroscope of any one of embodiments A18-A20, wherein the air cleaning device comprises an air cleaning filter.

A22. The slit lamp biomicroscope of any one of embodiments A18-A21, wherein the air cleaning device comprises a fan.

A23. The slit lamp biomicroscope of any one of embodiments A18-A22, wherein the air cleaning device comprises an air cleaning filter device.

A24. The slit lamp biomicroscope of embodiment A23, wherein the air cleaning filter device comprises an air cleaning filter and a fan.

A25. The slit lamp biomicroscope of any one of embodiments A18-A24, wherein the air cleaning filter is a HEPA filter.

A26. The slit lamp biomicroscope of any one of embodiments A18-A25, wherein the air cleaning device is positioned and configured for transferring a flow of air.

A27. The slit lamp biomicroscope of any one of embodiments A18-A26, wherein the air cleaning device transfers the flow of air by providing a flow of air through the air cleaning screen and emits the air outflow between the headrest and the biomicroscope.

A28. The slit lamp biomicroscope of any one of embodiments A18-A27, wherein the air cleaning device transfers the flow of air by providing a suction of air from an area between the headrest and the biomicroscope into the air cleaning screen.

A29. The slit lamp biomicroscope of any one of embodiments A18-A28, wherein the air cleaning device is further configured with the air cleaning filter.

A30. The slit lamp biomicroscope of embodiment A29, wherein the air cleaning filter is a HEPA filter.

A31. The slit lamp biomicroscope of any one of embodiments A18-A30, wherein the air cleaning device is further configured with the fan.

A32. The slit lamp biomicroscope of any one of embodiments A18-A31, wherein the air cleaning device is connected to the slit lamp biomicroscope proximate to the light tower.

A33. The slit lamp biomicroscope of any one of embodiments A18-A32, wherein the air cleaning device is connected to the slit lamp biomicroscope proximate to the headrest.

A34. The slit lamp biomicroscope of any one of embodiments A18-A33, wherein the air cleaning device is connected to the headrest, the biomicroscope base, or the table support.

A35. The slit lamp biomicroscope of any one of embodiments A18-A34, wherein the air cleaning device is mounted on a stand associated with the slit lamp biomicroscope.

A36. The slit lamp biomicroscope of any one of embodiments A18-A35, wherein the air cleaning device comprising the air cleaning screen and the air cleaning filter is mounted on a stand associated with the slit lamp biomicroscope.

A37. The slit lamp biomicroscope of any one of embodiments A18-A36, wherein the air cleaning screen is configured to be connected to the air cleaning filter device by a tube.

A38. The slit lamp biomicroscope of any one of embodiments A18-A37, wherein air cleaning filter device comprises the air cleaning filter and the fan.

A39. The slit lamp biomicroscope of any one of embodiments A18-A38, wherein the air cleaning filter is a HEPA filter.

A40. The slit lamp biomicroscope of any one of embodiments A18-A39, wherein the air cleaning filter excludes (filters) particles having a diameter of about 0.01-0.20 um or greater, such as particles having a diameter of about 0.05-0.15 um or greater.

A41. The slit lamp biomicroscope of any one of embodiments A18-A40, wherein the air cleaning filter device further comprises a UVC device.

A42. The slit lamp biomicroscope of any one of embodiments A1-A41, wherein the laminar airflow device is attached to the slit lamp biomicroscope via a support bracket.

A43. The slit lamp biomicroscope of any one of embodiments A1-A42, wherein the laminar airflow device support bracket is attached to the light tower.

A44. The slit lamp biomicroscope of any one of embodiments A1-A43, wherein the laminar airflow device support bracket is attached to the top of the light tower.

A45. The slit lamp biomicroscope of any one of embodiments A1-A43, wherein the laminar airflow device support bracket is attached to a side of the light tower.

A46. The slit lamp biomicroscope of any one of embodiments A1-A43, wherein the laminar airflow device support bracket is attached to the headrest.

A47. The slit lamp biomicroscope of any one of embodiments A1-A43, wherein the laminar airflow device is attached to a stand alone mount separate from the slit lamp biomicroscope.

A48. The slit lamp biomicroscope of any one of embodiments A1-A47, wherein the slit lamp biomicroscope can only be turned on if the laminar airflow device is turned on.

A49. The slit lamp biomicroscope of any one of embodiments A1-A48, wherein the slit lamp biomicroscope turns off if the laminar airflow device is turned off.

A50. The slit lamp biomicroscope of any one of embodiments A1-A49, wherein the laminar airflow device further comprises an laminar airflow generator.

A51. The slit lamp biomicroscope of any one of embodiments A1-A50, wherein the laminar airflow generator is a remote laminar airflow generator.

A52. The slit lamp biomicroscope of any one of embodiments A1-A51, wherein the laminar airflow generator is a quite laminar airflow generator.

A53. The slit lamp biomicroscope of any one of embodiments A1-A52, wherein the laminar airflow generator is a filtered air laminar airflow generator.

A54. The slit lamp biomicroscope of any one of embodiments A1-A53, wherein the laminar airflow emitted from the laminar airflow device is filtered air.

A55. The slit lamp biomicroscope of embodiment A54, wherein the filtered laminar airflow emitted from the laminar airflow device excludes particles having a diameter of about 0.01-0.20 um or greater.

A56. The slit lamp biomicroscope of embodiment A54, wherein the filtered laminar airflow emitted from the laminar airflow device excludes particles having a diameter of about 0.05-0.15 um or greater.

A57. The slit lamp biomicroscope of any one of embodiments A1-A56, wherein the laminar airflow generator is a cold air laminar airflow generator.

A58. The slit lamp biomicroscope of any one of embodiments A1-A57, wherein the laminar airflow emitted from the laminar airflow device is cold air.

A59. The slit lamp biomicroscope of embodiment A58, wherein the cold laminar airflow emitted from the laminar airflow device descends below the microscope base.

A60. The slit lamp biomicroscope of embodiment A58 or embodiment A59, wherein the cold laminar airflow emitted from the laminar airflow device descends below the table support.

A61. The slit lamp biomicroscope of any one of embodiments A58-A60, wherein the cold laminar airflow emitted from the laminar airflow device descends and remains below the microscope base.

A62. The slit lamp biomicroscope of any one of embodiments A58-A61, wherein the cold laminar airflow emitted from the laminar airflow device descends and remains below the table support.

A63. The slit lamp biomicroscope of any one of embodiments A1-A62, wherein the laminar airflow device emits laminar air between an examiner and an examinee.

A64. The slit lamp biomicroscope of any one of embodiments A1-A63, wherein the laminar airflow device emits laminar air in a downward, vertical trajectory.

A65. The slit lamp biomicroscope of any one of embodiments A1-A64, wherein the laminar airflow device emits a plane of laminar air or a plurality of parallel planes of laminar air in a downward, vertical trajectory.

A66. The slit lamp biomicroscope of any one of embodiments A1-A65, wherein the laminar airflow device emits laminar air in a vertical trajectory, parallel to light rays emitted from the light tower.

A67. The slit lamp biomicroscope of any one of embodiments A1-A66, wherein the laminar airflow device emits laminar air in a downward, non-vertical trajectory.

A68. The slit lamp biomicroscope of any one of embodiments A1-A67, wherein the laminar airflow device emits a plane of laminar air, a plurality of parallel planes of laminar air, or a plurality of non-parallel planes of laminar air, in a downward, non-vertical trajectory.

A69. The slit lamp biomicroscope of any one of embodiments A1-A63 or embodiments A67-A68, wherein the laminar airflow device emits laminar air in a non-vertical trajectory and non-parallel to light rays emitted from the light tower.

A70. The slit lamp biomicroscope of any one of embodiments A1-A69, wherein the airflow emitted from the laminar airflow device is emitted at a rate in the range of 0.3-0.7 m/s.

A71. The slit lamp biomicroscope of any one of embodiments A1-A70, wherein the airflow emitted from the laminar airflow device is emitted at a rate in the range of 0.3-0.5 m/s.

A72. The slit lamp biomicroscope of any one of embodiments A1-A71, wherein the airflow emitted from the laminar airflow device is emitted at a rate of 0.4 m/s.

A73. The slit lamp biomicroscope of any one of embodiments A1-A72, wherein the laminar airflow device comprises one or more exit ports from which air is emitted in a laminar manner.

A74. The slit lamp biomicroscope of embodiment A73, wherein the one or more exit ports is a series of exit ports from which air is emitted in a laminar manner.

A75. The slit lamp biomicroscope of embodiment A74, wherein the series of exit ports extend along the length of the laminar airflow device and emit air in a laminar manner in an identical, parallel, downward trajectory.

A76. The slit lamp biomicroscope of any one of embodiments A73-A75, wherein one or more exit ports is a single exit port extending along the length of the laminar airflow device from which air is emitted in a laminar manner.

A77. The slit lamp biomicroscope of any one of embodiments A73-A76, wherein the one or more exit ports comprises at least a first series of exit ports extending along the length of the laminar airflow device from which air is emitted in a laminar manner.

A78. The slit lamp biomicroscope of any one of embodiments A73-A77, wherein the one or more exit ports comprises at least a second series of exit ports extending along the length of the laminar airflow device from which air is emitted in a laminar manner.

A79. The slit lamp biomicroscope of embodiment A77 or embodiment A78, wherein the at least first series of exit ports and the at least second series of exit ports extending in parallel along the length of the laminar airflow device.

A80. The slit lamp biomicroscope of any one of embodiments A77-A79, wherein the laminar airflow emitted from the at least first series of exit ports and emitted from the at least second series of exit ports are in a parallel, downward vertical trajectory.

A81. The slit lamp biomicroscope of any one of embodiments A77-A80, wherein the laminar airflow emitted from the at least first series of exit ports is in a first downward non-vertical trajectory.

A82. The slit lamp biomicroscope of any one of embodiments A77-A81, wherein the laminar airflow emitted in the first downward non-vertical trajectory, is angled towards an examinee positioned at the headrest.

A83. The slit lamp biomicroscope of any one of embodiments A77-A82, wherein the laminar airflow emitted from the at least second series of exit ports is in a second downward non-vertical trajectory.

A84. The slit lamp biomicroscope of any one of embodiments A77-A83, wherein the laminar airflow emitted in the second downward non-vertical trajectory, is angled towards an examiner positioned looking through the microscope to examine an examinee positioned at the headrest.

A85. The slit lamp biomicroscope of any one of embodiments A77-A84, wherein the one or more exit ports is or comprises an exit port extending along the length of the laminar airflow device from which air is emitted in a laminar manner.

A86. The slit lamp biomicroscope of any one of embodiments A77-A85, wherein the one or more exit ports comprises at least a first exit port extending along the length of the laminar airflow device from which air is emitted in a laminar manner.

A87. The slit lamp biomicroscope of any one of embodiments A77-A86, wherein the one or more exit ports comprises at least a second exit port extending along the length of the laminar airflow device from which air is emitted in a laminar manner.

A88. The slit lamp biomicroscope of any one of embodiments A77-A87, wherein the laminar airflows emitted from the at least first and the at least second exit ports extending along the length of the laminar airflow device are in a parallel, downward vertical trajectory.

A89. The slit lamp biomicroscope of any one of embodiments A77-A88, wherein the laminar airflows emitted from the at least first and the at least second exit ports extending along the length of the laminar airflow device are in a first, downward non-vertical trajectory and a second, downward non-vertical trajectory, respectively.

A90. The slit lamp biomicroscope of any one of embodiments A77-A89, wherein the laminar airflow emitted in the first downward non-vertical trajectory, is angled towards an examinee positioned at the headrest.

A91. The slit lamp biomicroscope of any one of embodiments A77-A90, wherein the laminar airflow emitted in the second downward non-vertical trajectory, is angled towards an examiner positioned looking through the microscope to examine an examinee positioned at the headrest.

A92. The slit lamp biomicroscope of any one of embodiments A1-A91, wherein the laminar airflow device is a first laminar airflow device, and the slit lamp biomicroscope further comprises a second laminar airflow device.

A93. The slit lamp biomicroscope of embodiment A92, wherein the second laminar airflow device emits air in a laminar manner and in a downward, vertical trajectory.

A94. The slit lamp biomicroscope of embodiment A92 or embodiment A93, wherein the laminar airflow emitted from the second laminar airflow device is parallel to the laminar airflow emitted from the first laminar airflow device.

A95. The slit lamp biomicroscope of any one of embodiments A92-A94, wherein the laminar airflow emitted from the first laminar airflow device and the laminar airflow emitted from the second laminar airflow device are in a first downward, non-vertical trajectory and in a second downward, non-vertical trajectory, respectively.

A96. The slit lamp biomicroscope of any one of embodiments A92-A95, wherein the laminar airflow in the first downward, non-vertical trajectory is angled towards an examinee positioned at the headrest.

A97. The slit lamp biomicroscope of any one of embodiments A92-A96, wherein the laminar airflow in the second downward, non-vertical trajectory is angled towards an examiner positioned looking through the microscope to examine an examinee positioned at the headrest.

A98. The slit lamp biomicroscope of any one of embodiments A2-A97, wherein the drape or hood enclosure surrounding the slit lamp biomicroscope is transparent.

A99. The slit lamp biomicroscope of any one of embodiments A2-A98, wherein the drape or hood enclosure can be maneuvered to be pulled over and surround the slit lamp biomicroscope to prevent airborne particles from being moved between the examiner and the examinee.

A100. The slit lamp biomicroscope of any one of embodiments A2-A99, wherein the drape or hood enclosure can be maneuvered to be pulled over and surround the slit lamp biomicroscope to prevent contact contamination of the slit lamp biomicroscope.

A101. The slit lamp biomicroscope of any one of embodiments A2-A100, wherein the airborne particles comprise bacteria.

A102. The slit lamp biomicroscope of any one of embodiments A2-A101, wherein the airborne particles comprise viruses.

A103. The slit lamp biomicroscope of any one of embodiments A2-A102, wherein the drape or hood enclosure comprise an opening for an examiner's access to the slit lamp biomicroscope.

A104. The slit lamp biomicroscope of any one of embodiments A2-A103, wherein the drape or hood enclosure comprise an opening for an examinee's face to access to the headrest of the slit lamp biomicroscope.

A105. The slit lamp biomicroscope of any one of embodiments A2-A104, wherein the drape or hood enclosure comprises one opening for an examinee's eyes to access the enclosed slit lamp biomicroscope and a second opening for the examinee's nose and mouth to access the enclosed slit lamp biomicroscope.

A106. The slit lamp biomicroscope of any one of embodiments A2-A105, wherein the drape or hood enclosed slit lamp biomicroscope is connected to a suction pump having a filter to remove particles within said enclosure.

A107. The slit lamp biomicroscope of embodiment A106, wherein the filter is a HEPA filter.

A108. The slit lamp biomicroscope of embodiment A106 or embodiment A107, wherein the particles comprise microbes, bacteria and/or viruses.

A109. A method of using a slit lamp biomicroscope, comprising conducting an ophthalmologic examination using the slit lamp biomicroscope of any one of embodiments A1-A108.

A110. The method of use of embodiment A109, wherein the downward laminar airflow emitted from the laminar airflow device mitigates cross contamination of exhaled air from an examinee and an examiner traversing the slit lamp biomicroscope.

A111. The method of use of embodiment A109 or embodiment A110, wherein the downward laminar airflow emitted from the laminar airflow device blocks exhaled air from an examinee and an examiner traversing the slit lamp biomicroscope.

A112. The method of use of any one of embodiments A109-A111, wherein the downward laminar airflow emitted from the laminar airflow device minimizes the risk of being exposed to breath-laden contaminants adhering to the slit lamp biomicroscope.

A113. The method of use of any one of embodiments A109-A112, wherein the method reduces virus exposure to the subject examined during the ophthalmologic examination.

A114. The method of use of any one of embodiments A109-A113, wherein the method reduces virus exposure to the examiner utilizing the slit lamp biomicroscope to conduct the examination of the subject.

A115. The method of use of any one of embodiments A109-A114, wherein the method mitigates the risk of spreading a viral infection between a subject being examined and an examiner utilizing the slit lamp biomicroscope to conduct the examination.

A116. The method of use of any one of embodiments A109-A115, wherein the method mitigates the risk of contracting a viral infection between a subject being examined and an examiner utilizing the slit lamp biomicroscope to conduct the examination.

A117. The method of use of any one of embodiments A109-A116, wherein the virus is a coronavirus.

A118. The method of use of embodiment A117, wherein the coronavirus is SARS.

A119. The method of use of embodiment A117, wherein the coronavirus is COVID-19.

A120. The method of use of any one of embodiments A109-A119, wherein the virus is a rhinovirus.

A121. The method of use of embodiment A120, wherein the rhinovirus is influenza.

A122. The method of use of any one of embodiments A109-A121, wherein the downward laminar airflow emitted from the laminar airflow device mitigates cross contamination of microbes from an examinee and an examiner traversing the slit lamp biomicroscope.

A123. The method of use of any one of embodiments A109-A122, wherein the downward laminar airflow emitted from the laminar airflow device mitigates cross contamination of bacteria from an examinee and an examiner traversing the slit lamp biomicroscope.

A124. The method of use of any one of embodiments A109-A123, wherein the downward laminar airflow emitted from the laminar airflow device mitigates cross contamination of viruses from an examinee and an examiner traversing the slit lamp biomicroscope.

A125. The method of use of any one of embodiments A109-A124, wherein the method reduces microbe exposure to the subject examined during the ophthalmologic examination, and/or reduces microbe exposure to the examiner utilizing the slit lamp biomicroscope to conduct the examination of the subject.

A126. The method of use of any one of embodiments A109-A125, wherein the method reduces bacteria exposure to the subject examined during the ophthalmologic examination, and/or reduces bacteria exposure to the examiner utilizing the slit lamp biomicroscope to conduct the examination of the subject.

A127. The method of use of any one of embodiments A109-A126, wherein the method reduces virus exposure to the subject examined during the ophthalmologic examination, and/or reduces virus exposure to the examiner utilizing the slit lamp biomicroscope to conduct the examination of the subject.

A128. The method of use of any one of embodiments A109-A127, wherein the method mitigates the risk of spreading microbes between a subject being examined and an examiner utilizing the slit lamp biomicroscope to conduct the examination.

A129. The method of use of any one of embodiments A109-A128, wherein the method mitigates the risk of spreading bacteria between a subject being examined and an examiner utilizing the slit lamp biomicroscope to conduct the examination.

A130. The method of use of any one of embodiments A109-A129, wherein the method mitigates the risk of spreading viruses between a subject being examined and an examiner utilizing the slit lamp biomicroscope to conduct the examination.

A131. The method of use of any one of embodiments A109-A130, wherein the virus is a coronavirus.

A132. The method of use of embodiment A131, wherein the coronavirus is SARS.

A133. The method of use of embodiment A131, wherein the coronavirus is COVID-19.

A134. The method of use of any one of embodiments A109-A130, wherein the virus is a rhinovirus.

A135. The method of use of embodiment A134, wherein the rhinovirus is influenza.

### REFERENCES

1. http://www.frankshospitalworkshop.com/equipment/documents/ophthalmology/equipment/Eye%20Examination%20with%20the%20Slit%20Lamp%20%20Carl%20Zeiss.pdf accessed 7 Mar. 2020.

2. SARWAR M. A breath shield for ophthalmoscopy. Lancet. 1955; 269(6891):650. doi:10.1016/s0140-6736(55)92488-0

3. https://www.healio.com/ophthalmology/practice-management/news/online/%7Bd50a08e1-c1ac-43f8-90a1-c7207b9f2747%7D/anticipating-coronavirus-impacts-on-private-practice-a-thought-experiment. accessed 7 Mar. 2020.

4. Baud D, Qi X, Nielsen-Saines K, Musso D, Pomar L, Favre G. Real estimates of mortality following COVID-19 infection. Lancet Infect Dis. 2020 Mar. 12: S1473-3099(20)30195-X. doi: 10.1016/S1473-3099(20)30195-X. Epub ahead of print. PMID: 32171390.

5. https://www.enttoday.org/article/otolaryngologists-may-contract-covid-19-during-surgery/

6. https://www.bloomberg.com/news/articles/2020-03-17/europe-s-doctors-getting-sick-like-in-wuhan-chinese-doctors-say?fbclid=IwAR2ds9OWRxQuMHAuy5Gb7ltqUGMZNSojVNtFmq3zzcSLb_bO9aGYr7URxaI

7. van Doremalen N, Bushmaker T, Morris D H, Holbrook M G, Gamble A, Williamson B N, Tamin A, Harcourt J L, Thornburg N J, Gerber S I, Lloyd-Smith J O, de Wit E, Munster V J. Aerosol and Surface Stability of SARS-CoV-2 as Compared with SARS-CoV-1. N Engl J Med. 2020 Mar. 17. doi: 10.1056/NEJMc2004973. Epub ahead of print. PMID: 32182409.

8. Yan J, Grantham M, Pantelic J, et al. Infectious virus in exhaled breath of symptomatic seasonal influenza cases from a college community. Proc Natl Acad Sci USA. 2018; 115(5):1081-1086.

9. Sobolewska B, Buhl M, Liese J, Ziemssen F. Slit lamps and lenses: a potential source of nosocomial infections? Eye (Lond). 2018 June; 32(6):1021-1027. doi: 10.1038/s41433-017-0004-0. Epub 2018 Jan. 30. PMID: 29379104; PMCID: PMC5997676.

10. OYong K, Killerby M, Pan C Y, Huynh T, Green N M, Wadford D A, Terashita D. Outbreak of Epidemic Keratoconjunctivitis Caused by Human Adenovirus Type D53 in an Eye Care Clinic—Los Angeles County, 2017. MMWR Morb Mortal Wkly Rep. 2018 Dec. 7; 67(48):1347-1349. doi: 10.15585/mmwr.mm6748a4. PMID: 30521501; PMCID: PMC6329482.

11. Eye hospital eye. Lancet. 1983 Nov. 5; 2(8358):1065-6. PMID: 6138606.

12. Chams H, Mohammadi S F, Moayyeri A. Frequency and assortment of self-report occupational complaints among Iranian ophthalmologists: a preliminary survey. MedGenMed. 2004 Dec. 13; 6(4):1. PMID: 15775828; PMCID: PMC1480566.

13. Chan W M, Liu D T, Chan P K, et al. Precautions in ophthalmic practice in a hospital with a major acute SARS outbreak: an experience from Hong Kong. Eye (Lond). 2006; 20(3):283-289.

14. Li J O, Lam D S C, Chen Y, Ting D S W. Novel Coronavirus disease 2019 (COVID-19): The importance of recognising possible early ocular manifestation and using protective eyewear. Br J Ophthalmol. 2020 March; 104(3):297-298. doi: 10.1136/bjophthalmol-2020-315994. PMID: 32086236.

15. Coroneo M T. U.S. Application 63/003,518, filed Apr. 1, 2020.

16. Maxcy K F. The transmission of infection through the eye. JAMA. 1919; 72:636-639. doi:10.1001/jama.1919.02610090020005.

17. Flugge C. Uber Luftinfection. Z Hyg Infektionskr. 1897; 25:179-224.

18. Hare R. (1964) The transmission of respiratory infections, Proc. Roy. Soc. Med, 57, 221-230.

19. Liu L, Li Y, Nielsen P V, Wei J, Jensen R L (2016) Short-range airborne transmission of expiratory droplets between two people. Indoor Air 27:452-462.

20. Xie X., Li Y., Chwang A. T., Ho P. L. and Seto W. H. (2007) How far droplets can move in indoor environments—revising the Wells evaporation-falling curve, Indoor Air, 17, 211-225.

21. Wells W. F. (1934). On air-borne infection. Study II. Droplets and droplet nuclei. Am J Hyg, 20, 611-618.

22. Nicas M., Nazaroff W. W., and Hubbard A. (2005) Toward understanding the risk of secondary airborne infection: emission of respirable pathogen, J. Occup. Environ. Hyg., 2, 143-154.

23. Bjorn, E. and Nielsen, P. V. (2002). Dispersal of exhaled air and personal exposure in displacement ventilated rooms. Indoor Air, 12(3), 147-164.

24. Bischoff P, Kubilay N Z, Allegranzi B, Egger M, Gastmeier P. Effect of laminar airflow ventilation on surgical site infections: a systematic review and meta-analysis. Lancet Infect Dis. 2017 May; 17(5):553-561. doi: 10.1016/S1473-3099(17)30059-2. Epub 2017 Feb. 17. PMID: 28216243.

25. Whyte W, Lytsy B. Ultraclean air systems and the claim that laminar airflow systems fail to prevent deep infections after total joint arthroplasty. J Hosp Infect. 2019 September; 103(1):e9-e15. doi: 10.1016/j.jhin.2019.04.021. Epub 2019 May 3. PMID: 31059724.

26. https://surgicube.com/efficiency-of-surgicube-airflow-device-to-prevent-covid-19-2/ accessed 23 Mar. 2020.

27. Chu C M, Poon L L, Cheng V C, Chan K S, Hung I F, Wong M M, Chan K H, Leung W S, Tang B S, Chan V L, Ng W L, Sim T C, Ng P W, Law K I, Tse D M, Peiris J S, Yuen K Y. Initial viral load and the outcomes of SARS. CMAJ. 2004 Nov. 23; 171(11):1349-52. doi: 10.1503/cmaj.1040398. PMID: 15557587; PMCID: PMC527336.

### INCORPORATION BY REFERENCE

Various references such as patents, patent applications, and publications are cited herein, the disclosures of which are hereby incorporated by reference herein in their entireties.

We claim:

1. A slit lamp biomicroscope, comprising:
i) a microscope;
ii) a light tower; and
iii) a laminar airflow device.

2. A slit lamp biomicroscope, comprising:
i) a microscope;
ii) a light tower; and
iii) an air cleaning device; and
iv) a drape or hood enclosure surrounding the slit lamp biomicroscope.

3. The slit lamp biomicroscope of claim 1 or claim 2, wherein the slit lamp biomicroscope further comprises a mirror.

4. The slit lamp biomicroscope of any one of claims 1-3, wherein the slit lamp biomicroscope further comprises a headrest.

5. The slit lamp biomicroscope of claim 4, wherein the headrest is suitable for a patient to place their head against during an eye examination or procedure.

6. The slit lamp biomicroscope of any one of claims 1-5, wherein the slit lamp biomicroscope further comprises a biomicroscope base.

7. The slit lamp biomicroscope of claim 6, wherein the biomicroscope base comprises one or more deflectors.

8. The slit lamp biomicroscope of claim 6 or claim 7, wherein the biomicroscope base comprises one or more air aspirators.

9. The slit lamp biomicroscope of any one of claims 1-8, wherein the slit lamp biomicroscope is mounted to a table support.

10. The slit lamp biomicroscope of claim 9, wherein the table support comprises one or more deflectors.

11. The slit lamp biomicroscope of claim 9 or claim 10, wherein the table support comprises one or more air aspirators.

12. The slit lamp biomicroscope of any one of claims 7-11, wherein the one or more deflectors deflect laminar airflow emitted from the laminar airflow device.

13. The slit lamp biomicroscope of any one of claims 7-12, wherein the laminar airflow deflected by the one or more deflectors is angled towards an examinee positioned at the headrest.

14. The slit lamp biomicroscope of any one of claims 7-13, wherein the laminar airflow deflected by the one or more deflectors is angled towards an examiner positioned looking through the microscope to examine an examinee positioned at the headrest.

15. The slit lamp biomicroscope of any one of claims 7-14, wherein a first portion of the deflected laminar airflow is angled towards an examinee positioned at the headrest, and a second portion of the deflected laminar airflow is angled towards an examiner positioned looking through the microscope to examine an examinee positioned at the headrest.

16. The slit lamp biomicroscope of any one of claims 8-15, wherein the one or more air aspirators remove the emitted laminar airflow.

17. The slit lamp biomicroscope of any one of claims 8-16, wherein the one or more air aspirators remove the emitted laminar airflow and transmit it towards a filtering device.

18. The slit lamp biomicroscope of any one of claim 1 or claims 3-17, wherein the slit lamp biomicroscope further comprises an air cleaning device.

19. The slit lamp biomicroscope of claim 18, wherein the air cleaning device is located proximate to the headrest.

20. The slit lamp biomicroscope of claim 18 or claim 19, wherein the air cleaning device comprises an air cleaning screen.

21. The slit lamp biomicroscope of any one of claims 18-20, wherein the air cleaning device comprises an air cleaning filter.

22. The slit lamp biomicroscope of any one of claims 18-21, wherein the air cleaning device comprises a fan.

23. The slit lamp biomicroscope of any one of claims 18-22, wherein the air cleaning device comprises an air cleaning filter device.

24. The slit lamp biomicroscope of claim 23, wherein the air cleaning filter device comprises an air cleaning filter and a fan.

25. The slit lamp biomicroscope of any one of claims 18-24, wherein the air cleaning filter is a HEPA filter.

26. The slit lamp biomicroscope of any one of claims 18-25, wherein the air cleaning device is positioned and configured for transferring a flow of air.

27. The slit lamp biomicroscope of any one of claims 18-26, wherein the air cleaning device transfers the flow of air by providing a flow of air through the air cleaning screen and emits the air outflow between the headrest and the biomicroscope.

28. The slit lamp biomicroscope of any one of claims 18-27, wherein the air cleaning device transfers the flow of air by providing a suction of air from an area between the headrest and the biomicroscope into the air cleaning screen.

29. The slit lamp biomicroscope of any one of claims 18-28, wherein the air cleaning device is further configured with the air cleaning filter.

30. The slit lamp biomicroscope of claim 29, wherein the air cleaning filter is a HEPA filter.

31. The slit lamp biomicroscope of any one of claims 18-30, wherein the air cleaning device is further configured with the fan.

32. The slit lamp biomicroscope of any one of claims 18-31, wherein the air cleaning device is connected to the slit lamp biomicroscope proximate to the light tower.

33. The slit lamp biomicroscope of any one of claims 18-32, wherein the air cleaning device is connected to the slit lamp biomicroscope proximate to the headrest.

34. The slit lamp biomicroscope of any one of claims 18-33, wherein the air cleaning device is connected to the headrest, the biomicroscope base, or the table support.

35. The slit lamp biomicroscope of any one of claims 18-34, wherein the air cleaning device is mounted on a stand associated with the slit lamp biomicroscope.

36. The slit lamp biomicroscope of any one of claims 18-35, wherein the air cleaning device comprising the air cleaning screen and the air cleaning filter is mounted on a stand associated with the slit lamp biomicroscope.

37. The slit lamp biomicroscope of any one of claims 18-36, wherein the air cleaning screen is configured to be connected to the air cleaning filter device by a tube.

38. The slit lamp biomicroscope of any one of claims 18-37, wherein air cleaning filter device comprises the air cleaning filter and the fan.

39. The slit lamp biomicroscope of any one of claims 18-38, wherein the air cleaning filter is a HEPA filter.

40. The slit lamp biomicroscope of any one of claims 18-39, wherein the air cleaning filter excludes (filters) particles having a diameter of about 0.01-0.20 um or greater, such as particles having a diameter of about 0.05-0.15 um or greater.

41. The slit lamp biomicroscope of any one of claims 18-40, wherein the air cleaning filter device further comprises a UVC device.

42. The slit lamp biomicroscope of any one of claims 1-41, wherein the laminar airflow device is attached to the slit lamp biomicroscope via a support bracket.

43. The slit lamp biomicroscope of any one of claims 1-42, wherein the laminar airflow device support bracket is attached to the light tower.

44. The slit lamp biomicroscope of any one of claims 1-43, wherein the laminar airflow device support bracket is attached to the top of the light tower.

45. The slit lamp biomicroscope of any one of claims 1-43, wherein the laminar airflow device support bracket is attached to a side of the light tower.

46. The slit lamp biomicroscope of any one of claims 1-43, wherein the laminar airflow device support bracket is attached to the headrest.

47. The slit lamp biomicroscope of any one of claims 1-43, wherein the laminar airflow device is attached to a stand alone mount separate from the slit lamp biomicroscope.

48. The slit lamp biomicroscope of any one of claims 1-47, wherein the slit lamp biomicroscope can only be turned on if the laminar airflow device is turned on.

49. The slit lamp biomicroscope of any one of claims 1-48, wherein the slit lamp biomicroscope turns off if the laminar airflow device is turned off.

50. The slit lamp biomicroscope of any one of claims 1-49, wherein the laminar airflow device further comprises an laminar airflow generator.

51. The slit lamp biomicroscope of any one of claims 1-50, wherein the laminar airflow generator is a remote laminar airflow generator.

52. The slit lamp biomicroscope of any one of claims 1-51, wherein the laminar airflow generator is a quite laminar airflow generator.

53. The slit lamp biomicroscope of any one of claims 1-52, wherein the laminar airflow generator is a filtered air laminar airflow generator.

54. The slit lamp biomicroscope of any one of claims 1-53, wherein the laminar airflow emitted from the laminar airflow device is filtered air.

55. The slit lamp biomicroscope of claim 54, wherein the filtered laminar airflow emitted from the laminar airflow device excludes particles having a diameter of about 0.01-0.20 um or greater.

56. The slit lamp biomicroscope of claim 54, wherein the filtered laminar airflow emitted from the laminar airflow device excludes particles having a diameter of about 0.05-0.15 um or greater.

57. The slit lamp biomicroscope of any one of claims 1-56, wherein the laminar airflow generator is a cold air laminar airflow generator.

58. The slit lamp biomicroscope of any one of claims 1-57, wherein the laminar airflow emitted from the laminar airflow device is cold air.

59. The slit lamp biomicroscope of claim 58, wherein the cold laminar airflow emitted from the laminar airflow device descends below the microscope base.

60. The slit lamp biomicroscope of claim 58 or claim 59, wherein the cold laminar airflow emitted from the laminar airflow device descends below the table support.

61. The slit lamp biomicroscope of any one of claims 58-60, wherein the cold laminar airflow emitted from the laminar airflow device descends and remains below the microscope base.

62. The slit lamp biomicroscope of any one of claims 58-61, wherein the cold laminar airflow emitted from the laminar airflow device descends and remains below the table support.

63. The slit lamp biomicroscope of any one of claims 1-62, wherein the laminar airflow device emits laminar air between an examiner and an examinee.

64. The slit lamp biomicroscope of any one of claims 1-63, wherein the laminar airflow device emits laminar air in a downward, vertical trajectory.

65. The slit lamp biomicroscope of any one of claims 1-64, wherein the laminar airflow device emits a plane of laminar air or a plurality of parallel planes of laminar air in a downward, vertical trajectory.

66. The slit lamp biomicroscope of any one of claims 1-65, wherein the laminar airflow device emits laminar air in a vertical trajectory, parallel to light rays emitted from the light tower.

67. The slit lamp biomicroscope of any one of claims 1-66, wherein the laminar airflow device emits laminar air in a downward, non-vertical trajectory.

68. The slit lamp biomicroscope of any one of claims 1-67, wherein the laminar airflow device emits a plane of laminar air, a plurality of parallel planes of laminar air, or a plurality of non-parallel planes of laminar air, in a downward, non-vertical trajectory.

69. The slit lamp biomicroscope of any one of claims 1-63 or claims 67-68, wherein the laminar airflow device emits laminar air in a non-vertical trajectory and non-parallel to light rays emitted from the light tower.

70. The slit lamp biomicroscope of any one of claims 1-69, wherein the airflow emitted from the laminar airflow device is emitted at a rate in the range of 0.3-0.7 m/s.

71. The slit lamp biomicroscope of any one of claims 1-70, wherein the airflow emitted from the laminar airflow device is emitted at a rate in the range of 0.3-0.5 m/s.

72. The slit lamp biomicroscope of any one of claims 1-71, wherein the airflow emitted from the laminar airflow device is emitted at a rate of 0.4 m/s.

73. The slit lamp biomicroscope of any one of claims 1-72, wherein the laminar airflow device comprises one or more exit ports from which air is emitted in a laminar manner.

74. The slit lamp biomicroscope of claim 73, wherein the one or more exit ports is a series of exit ports from which air is emitted in a laminar manner.

75. The slit lamp biomicroscope of claim 74, wherein the series of exit ports extend along the length of the laminar airflow device and emit air in a laminar manner in an identical, parallel, downward trajectory.

76. The slit lamp biomicroscope of any one of claims 73-75, wherein one or more exit ports is a single exit port extending along the length of the laminar airflow device from which air is emitted in a laminar manner.

77. The slit lamp biomicroscope of any one of claims 73-76, wherein the one or more exit ports comprises at least a first series of exit ports extending along the length of the laminar airflow device from which air is emitted in a laminar manner.

78. The slit lamp biomicroscope of any one of claims 73-77, wherein the one or more exit ports comprises at least a second series of exit ports extending along the length of the laminar airflow device from which air is emitted in a laminar manner.

79. The slit lamp biomicroscope of claim 77 or claim 78, wherein the at least first series of exit ports and the at least second series of exit ports extending in parallel along the length of the laminar airflow device.

80. The slit lamp biomicroscope of any one of claims 77-79, wherein the laminar airflow emitted from the at least first series of exit ports and emitted from the at least second series of exit ports are in a parallel, downward vertical trajectory.

81. The slit lamp biomicroscope of any one of claims 77-80, wherein the laminar airflow emitted from the at least first series of exit ports is in a first downward non-vertical trajectory.

82. The slit lamp biomicroscope of any one of claims 77-81, wherein the laminar airflow emitted in the first downward non-vertical trajectory, is angled towards an examinee positioned at the headrest.

83. The slit lamp biomicroscope of any one of claims 77-82, wherein the laminar airflow emitted from the at least second series of exit ports is in a second downward non-vertical trajectory.

84. The slit lamp biomicroscope of any one of claims 77-83, wherein the laminar airflow emitted in the second downward non-vertical trajectory, is angled towards an examiner positioned looking through the microscope to examine an examinee positioned at the headrest.

85. The slit lamp biomicroscope of any one of claims 77-84, wherein the one or more exit ports is or comprises an exit port extending along the length of the laminar airflow device from which air is emitted in a laminar manner.

86. The slit lamp biomicroscope of any one of claims 77-85, wherein the one or more exit ports comprises at least a first exit port extending along the length of the laminar airflow device from which air is emitted in a laminar manner.

87. The slit lamp biomicroscope of any one of claims 77-86, wherein the one or more exit ports comprises at least a second exit port extending along the length of the laminar airflow device from which air is emitted in a laminar manner.

88. The slit lamp biomicroscope of any one of claims 77-87, wherein the laminar airflows emitted from the at least first and the at least second exit ports extending along the length of the laminar airflow device are in a parallel, downward vertical trajectory.

89. The slit lamp biomicroscope of any one of claims 77-88, wherein the laminar airflows emitted from the at least first and the at least second exit ports extending along the length of the laminar airflow device are in a first, downward non-vertical trajectory and a second, downward non-vertical trajectory, respectively.

90. The slit lamp biomicroscope of any one of claims 77-89, wherein the laminar airflow emitted in the first downward non-vertical trajectory, is angled towards an examinee positioned at the headrest.

91. The slit lamp biomicroscope of any one of claims 77-90, wherein the laminar airflow emitted in the second downward non-vertical trajectory, is angled towards an examiner positioned looking through the microscope to examine an examinee positioned at the headrest.

92. The slit lamp biomicroscope of any one of claims 1-91, wherein the laminar airflow device is a first laminar airflow device, and the slit lamp biomicroscope further comprises a second laminar airflow device.

93. The slit lamp biomicroscope of claim 92, wherein the second laminar airflow device emits air in a laminar manner and in a downward, vertical trajectory.

94. The slit lamp biomicroscope of claim 92 or claim 93, wherein the laminar airflow emitted from the second laminar airflow device is parallel to the laminar airflow emitted from the first laminar airflow device.

95. The slit lamp biomicroscope of any one of claims 92-94, wherein the laminar airflow emitted from the first laminar airflow device and the laminar airflow emitted from the second laminar airflow device are in a first downward, non-vertical trajectory and in a second downward, non-vertical trajectory, respectively.

96. The slit lamp biomicroscope of any one of claims 92-95, wherein the laminar airflow in the first downward, non-vertical trajectory is angled towards an examinee positioned at the headrest.

97. The slit lamp biomicroscope of any one of claims 92-96, wherein the laminar airflow in the second downward, non-vertical trajectory is angled towards an examiner positioned looking through the microscope to examine an examinee positioned at the headrest.

98. The slit lamp biomicroscope of any one of claims 2-97, wherein the drape or hood enclosure surrounding the slit lamp biomicroscope is transparent.

99. The slit lamp biomicroscope of any one of claims 2-98, wherein the drape or hood enclosure can be maneuvered to be pulled over and surround the slit lamp biomicroscope to prevent airborne particles from being moved between the examiner and the examinee.

100. The slit lamp biomicroscope of any one of claims 2-99, wherein the drape or hood enclosure can be maneuvered to be pulled over and surround the slit lamp biomicroscope to prevent contact contamination of the slit lamp biomicroscope.

101. The slit lamp biomicroscope of any one of claims 2-100, wherein the airborne particles comprise bacteria.

102. The slit lamp biomicroscope of any one of claims 2-101, wherein the airborne particles comprise viruses.

103. The slit lamp biomicroscope of any one of claims 2-102, wherein the drape or hood enclosure comprise an opening for an examiner's access to the slit lamp biomicroscope.

104. The slit lamp biomicroscope of any one of claims 2-103, wherein the drape or hood enclosure comprise an opening for an examinee's face to access to the headrest of the slit lamp biomicroscope.

105. The slit lamp biomicroscope of any one of claims 2-104, wherein the drape or hood enclosure comprises one opening for an examinee's eyes to access the enclosed slit lamp biomicroscope and a second opening for the examinee's nose and mouth to access the enclosed slit lamp biomicroscope.

106. The slit lamp biomicroscope of any one of claims 2-105, wherein the drape or hood enclosed slit lamp biomicroscope is connected to a suction pump having a filter to remove particles within said enclosure.

107. The slit lamp biomicroscope of claim 106, wherein the filter is a HEPA filter.

108. The slit lamp biomicroscope of claim 106 or claim 107, wherein the particles comprise microbes, bacteria and/or viruses.

109. A method of using a slit lamp biomicroscope, comprising conducting an ophthalmologic examination using the slit lamp biomicroscope of any one of claims 1-108.

110. The method of use of claim 109, wherein the downward laminar airflow emitted from the laminar airflow device mitigates cross contamination of exhaled air from an examinee and an examiner traversing the slit lamp biomicroscope.

111. The method of use of claim 109 or claim 110, wherein the downward laminar airflow emitted from the laminar airflow device blocks exhaled air from an examinee and an examiner traversing the slit lamp biomicroscope.

112. The method of use of any one of claims 109-111, wherein the downward laminar airflow emitted from the laminar airflow device minimizes the risk of being exposed to breath-laden contaminants adhering to the slit lamp biomicroscope.

113. The method of use of any one of claims 109-112, wherein the method reduces virus exposure to the subject examined during the ophthalmologic examination.

114. The method of use of any one of claims 109-113, wherein the method reduces virus exposure to the examiner utilizing the slit lamp biomicroscope to conduct the examination of the subject.

115. The method of use of any one of claims 109-114, wherein the method mitigates the risk of spreading a viral infection between a subject being examined and an examiner utilizing the slit lamp biomicroscope to conduct the examination.

116. The method of use of any one of claims 109-115, wherein the method mitigates the risk of contracting a viral infection between a subject being examined and an examiner utilizing the slit lamp biomicroscope to conduct the examination.

117. The method of use of any one of claims 109-116, wherein the virus is a coronavirus.

118. The method of use of claim 117, wherein the coronavirus is SARS.

119. The method of use of claim 117, wherein the coronavirus is COVID-19.

120. The method of use of any one of claims 109-119, wherein the virus is a rhinovirus.

121. The method of use of claim 120, wherein the rhinovirus is influenza.
